(19) United States
(12) Patent Application Publication
(10) Pub. No.: US 2026/0208100 A1
(43) Pub. Date: Jul. 23, 2026

(54) CARBON DIOXIDE RECOVERY APPARATUS AND CARBON DIOXIDE RECOVERY METHOD

(71) Applicants: KANKYO KOUGAKU, LTD., Takasaki-shi, Gunma (JP); KANKYOU-SYSTEMS, INC., Takasaki-shi, Gunma (JP)

(72) Inventor: Kenji SORIMACHI, Takasaki-shi, Gunma (JP)

(21) Appl. No.: 19/138,298

(22) PCT Filed: Jun. 9, 2023
(86) PCT No.: PCT/JP2023/021554

§ 371 Date: Jun. 12, 2025

(30) Foreign Application Priority Data
Dec. 14, 2022 (JP) 2022-199049
Apr. 27, 2023 (JP) 2023-073210

(51) Int. Cl. B01D 53/62; B01D 53/78; B01D 53/96; C25B 1/22; C25B 1/46

(52) U.S. Cl. CPC B01D 53/62; B01D 53/78; B01D 53/965; C25B 1/22; C25B 1/46; B01D 2257/504

(57) ABSTRACT

A carbon dioxide recovery apparatus is configured to utilizes electrolysis and to help prevent chlorine gas generated during electrolysis from being discharged to the outside. A carbon dioxide recovery apparatus includes an electrolysis vessel, a reaction vessel. a liquid transfer part, and a gas transfer part. An anode electrolysis chamber and a cathode electrolysis chamber are provided in the electrolysis vessel, to which an aqueous metal salt solution is supplied. An aqueous metal hydroxide salt solution generated by electrolysis in the cathode electrolysis chamber and carbon dioxide are transferred to the reaction vessel, which react to form a carbonate. Chlorine gas generated in the anode electrolysis chamber is supplied to the aqueous metal salt solution in the anode electrolysis chamber in the electrolysis vessel by the gas transfer part.

### TECHNICAL FIELD

The present invention relates to a carbon dioxide recovery apparatus and a carbon dioxide recovery method.

### BACKGROUND ART

Patent Literature 1 discloses a technique for carbon dioxide fixation utilizing electrolysis. The system disclosed in Patent Literature 1 first electrolyzes an aqueous sodium chloride solution to generate an aqueous sodium hydroxide solution. Next, the system generates an aqueous solution containing sodium carbonate and sodium bicarbonate by supplying air containing carbon dioxide to the aqueous sodium hydroxide solution. The system is designed to extract carbon dioxide gas by supplying hydrochloric acid generated by electrolysis to the aqueous solution.

### CITATION LIST

### Patent Literature

Patent Literature 1: JP 7004881 B

### SUMMARY OF INVENTION

### Technical Problem

The system disclosed in Patent Literature 1 generates chlorine gas during electrolysis. A portion of the chlorine gas is dissolved in the aqueous solution to form hydrochloric acid and hypochlorous acid. On the other hand, the chlorine gas that has not been dissolved in the aqueous solution remains as chlorine gas. Therefore, a technical challenge with the aforementioned system is to prevent the remaining gas from being discharged outside the apparatus.

In view of the above, an object of the present disclosure is to provide a carbon dioxide recovery apparatus which utilizes electrolysis and can prevent chlorine gas generated during electrolysis from being discharged to the outside.

### Solution to Problem

In order to achieve the above object, the present disclosure provides a carbon dioxide recovery apparatus, including: an electrolysis vessel; a reaction vessel; a liquid transfer part; and a gas transfer part, wherein the electrolysis vessel includes a diaphragm, an anode, and a cathode, an anode electrolysis chamber and a cathode electrolysis chamber are provided in the electrolysis vessel by the diaphragm, the anode is disposed in the anode electrolysis chamber, the cathode is disposed in the cathode electrolysis chamber, the liquid transfer part is configured to supply an aqueous metal salt solution to both the anode electrolysis chamber and cathode electrolysis chamber, an aqueous metal hydroxide solution is generated by electrolysis in the cathode electrolysis chamber, the liquid transfer part is configured to transfer an aqueous metal hydroxide salt solution to the reaction vessel, the gas transfer part is configured to supply carbon dioxide-containing gas to the aqueous metal hydroxide salt solution in the reaction vessel, carbon dioxide in the carbon dioxide-containing gas reacts with a metal salt in the aqueous metal hydroxide salt solution to generate a carbonate, and the gas transfer part is configured to supply chlorine gas generated in the anode electrolysis chamber to the aqueous metal salt solution in the anode electrolysis chamber in the electrolysis vessel.

The present disclosure also provides a carbon dioxide recovery method, including: electrolyzing; reacting; and supplying chlorine gas, wherein the electrolyzing is performed using an electrolysis vessel, the electrolysis vessel includes a diaphragm, an anode, and a cathode, an anode electrolysis chamber and a cathode electrolysis chamber are provided in the electrolysis vessel by the diaphragm, the anode is disposed in the anode electrolysis chamber, the cathode is disposed in the cathode electrolysis chamber, in the electrolyzing, an aqueous metal salt solution is supplied to both the anode electrolysis chamber and the cathode electrolysis chamber, and an aqueous metal hydroxide solution is generated by electrolysis in the cathode electrolysis chamber, the reacting is performed using a reaction vessel, in the reacting, the aqueous metal hydroxide salt solution is transferred to the reaction vessel, carbon dioxide-containing gas is supplied to the aqueous metal hydroxide salt solution in the reaction vessel, and carbon dioxide in the carbon dioxide-containing gas reacts with a metal salt in the aqueous metal hydroxide salt solution to generate carbonate, and in the supplying chlorine gas, the chlorine gas generated in the anode electrolysis chamber is supplied to the aqueous metal salt solution in the anode electrolysis chamber in the electrolysis vessel.

### Advantageous Effects of Invention

According to the present disclosure, when carbon dioxide recovery is performed utilizing electrolysis, chlorine gas generated during electrolysis can be prevented from being discharged to the outside.

### BRIEF DESCRIPTION OF DRAWINGS

FIG. 1 is a first schematic diagram showing an example of the configuration of a carbon dioxide recovery apparatus according to the present disclosure.

FIG. 2 is a second schematic diagram showing an example of the configuration of the carbon dioxide recovery apparatus according to the present disclosure.

FIG. 3 is a third schematic diagram showing an example of the configuration of the carbon dioxide recovery apparatus according to the present disclosure.

FIG. 4 is a fourth schematic diagram showing an example of the configuration of the carbon dioxide recovery apparatus according to the present disclosure.

FIG. 5 is a fifth schematic diagram showing an example of the configuration of the carbon dioxide recovery apparatus according to the present disclosure.

FIG. 6 is a sixth schematic diagram showing an example of the configuration of the carbon dioxide recovery apparatus according to the present disclosure.

FIG. 7 is a seventh schematic diagram showing an example of the configuration of the carbon dioxide recovery apparatus according to the present disclosure.

FIG. 8 is an eighth schematic diagram showing an example of the configuration of the carbon dioxide recovery apparatus according to the present disclosure.

FIG. 9 is a ninth schematic diagram showing an example of the configuration of the carbon dioxide recovery apparatus according to the present disclosure.

FIG. 10 is a vertical cross-sectional view showing a specific example of the carbon dioxide recovery apparatus according to the present disclosure.

FIG. 11 is a vertical cross-sectional view showing another specific example of the carbon dioxide recovery apparatus according to the present disclosure.

### DESCRIPTION OF EMBODIMENTS

The apparatus and method according to the embodiments of the present disclosure will be described with reference to the drawings. The present disclosure is not limited to the following embodiments. In the following drawings, identical parts are indicated with identical reference signs. In addition, unless otherwise stated, the descriptions of the embodiments may be referred to for one another, and the configurations of the embodiments may be combined.

### First Embodiment

First, with reference to FIG. 1, an example of the configuration of the carbon dioxide recovery apparatus 1A of the present embodiment (hereinafter also referred to as the “apparatus 1A”) will be described. As shown in FIG. 1, the apparatus 1A includes, for example, an electrolysis vessel 2 and a reaction vessel 3. The electrolysis vessel 2 and the reaction vessel 3 are, for example, rectangular parallel piped containers as shown in FIG. 1, but are not limited thereto. Each of the vessels may have, for example, a cylindrical shape or any shape. Furthermore, the electrolysis vessel 2 and the reaction vessel 3 may either be provided independently or integrally. Furthermore, the apparatus 1A may include at least one electrolysis vessel 2 and at least one reaction vessel 3, and any number of electrolysis vessels 2 and reaction vessels 3 may be provided. The material of each vessel may be any material as long as electrolysis in the electrolysis vessel 2 and chemical reaction in the reaction vessel 3 are possible, and the material may be in any form.

The electrolysis vessel 2 includes a diaphragm 100, an anode 24, and a cathode 25. An anode electrolysis chamber 20 and a cathode electrolysis chamber 21 are provided in the electrolysis vessel 2 by the diaphragm 100. The diaphragm 100 may be, for example, a hard member, or a film-like member such as a semipermeable membrane. As such, the material of the diaphragm 100 is not limited. In addition, in FIG. 1, the diaphragm 100 extends to the bottom of the electrolysis vessel 2 to completely divide the electrolysis vessel 2, but is not limited thereto. For example, the diaphragm 100 may extend from the upper part of the electrolysis vessel 2 to the vicinity of the center, allowing the lower part of the electrolysis vessel 2 to be in fluid communication. The shape, position, material, and the like of the diaphragm 100 are not particularly limited as long as it can separate the anode electrolysis chamber 20 and the cathode electrolysis chamber 21 in the electrolysis vessel 2, electrolyze the aqueous solution in the electrolysis vessel 2, and produce the products in the anode electrolysis chamber 20 and the cathode electrolysis chamber 21, respectively. For example, the diaphragm 100, which partitions the electrolysis vessel 2 into right and left spaces, may be made of a hard impermeable material and its lower end may be opened to allow the anode electrolysis chamber 20 and the cathode electrolysis chamber 21 to be in fluid communication. Also, the diaphragm 100 may be formed of a semi-permeable membrane and may completely partition the right and left spaces of the electrolysis vessel 2. The anode 24 is disposed in the anode electrolysis chamber 20. The cathode 25 is disposed in the cathode electrolysis chamber 21.

As shown in FIG. 1, the apparatus 1A includes, for example, a liquid transfer part and a gas transfer part. The liquid transfer part is configured, for example, to supply an aqueous solution to each of the vessels such as the electrolysis vessel 2 and the reaction vessel 3, to discharge the aqueous solution from each vessel, and to transfer the aqueous solution from any vessel to a different vessel. Therefore, it is not always the case that a single liquid transfer part is provided, and, for example, two or more such parts, including a liquid transfer part 18 configured to supply the aqueous solution to the electrolysis vessel 2 and a liquid transfer part 16 configured to transfer the aqueous solution from the electrolysis vessel 2 to the reaction vessel 3 may be provided. The liquid transfer part 18 shown in FIG. 1 is configured, for example, to supply an aqueous metal salt solution to both the anode electrolysis chamber 20 and the cathode electrolysis chamber 21. The liquid transfer part 16 shown in FIG. 1 is configured, for example, to transfer an aqueous metal hydroxide salt solution from the electrolysis vessel 2 to the reaction vessel 3. Further, the liquid transfer part may be made of, for example, either a hard material or a soft material. The liquid transfer part may be, for example, pipe-shaped, but is not limited thereto. The material, shape, and the like of the liquid transfer part are not particularly limited as long as it is configured to transfer an aqueous solution. The liquid transfer part may be provided with, for example, a pump configured to transfer the aqueous solution. The gas transfer part is configured, for example, to supply gas to each vessel, to discharge gas from each vessel, and to transfer gas from any position to another position. Also, as similarly described for the liquid transfer part, it is not always the case that a single gas transfer part is provided, and two or more gas transfer parts may be provided. In FIG. 1, the gas transfer part includes, for example, a gas transfer part 19 configured to supply air to the reaction vessel 3 and a gas transfer part 22 configured to transfer gas from the upper part of the electrolysis vessel 2 into the aqueous solution in the lower part of the electrolysis vessel 2. The gas transfer part 19 shown in FIG. 1 is configured, for example, to supply carbon dioxide-containing gas into the aqueous metal hydroxide salt solution in the reaction vessel. Further, the gas transfer part 22 shown in FIG. 1 is configured, for example, to supply chlorine gas generated in the anode electrolysis chamber 20 into the aqueous metal salt solution in the anode electrolysis chamber 20 in the electrolysis vessel 2. As similarly described for the liquid transfer part, the material, shape, and the like of the gas transfer part are not particularly limited, as long as it is configured to transfer gas, and for example, the gas transfer part may be provided with a pump.

Next, an example of the carbon dioxide recovery method of the present embodiment will be described. The carbon dioxide recovery method of the present embodiment is performed as follows, for example, using the apparatus 1A shown in FIG. 1. The carbon dioxide recovery method of the present embodiment is not limited to the use of the apparatus 1A shown in FIG. 1. Further, the order in which the following processes are performed is not limited to the order described. Therefore, the carbon dioxide recovery method of the present embodiment may be performed, for example, in an order different from the order described, or two or more processes may be performed simultaneously.

First, in the electrolysis vessel 2, the liquid transfer part 18 supplies the aqueous metal salt solution to both the anode electrolysis chamber 20 and the cathode electrolysis chamber 21. Then, the electrolysis vessel 2 generates an aqueous metal hydroxide solution by electrolysis in the cathode electrolysis chamber 21 (electrolysis process). As described above, the diaphragm 100 of the electrolysis vessel 2 may, for example, not extend to the bottom surface of the electrolysis vessel 2. That is, the lower part of the electrolysis vessel 2 may be open to allow the anode electrolysis chamber 20 and the cathode electrolysis chamber 21 to be in fluid communication. Therefore, the liquid transfer part 18 is not always configured to inject the aqueous metal salt solution into two places. The liquid transfer part 18 may, for example, inject the aqueous metal salt solution into one place in the lower part of the electrolysis vessel 2, supplying the aqueous metal salt solution to both the anode electrolysis chamber 20 and the cathode electrolysis chamber 21 in the electrolysis vessel 2. Examples of the metal include alkali metals and alkaline earth metals. Examples of the aqueous metal salt solution include an aqueous sodium chloride solution, an aqueous potassium chloride solution, an aqueous calcium chloride solution, and an aqueous magnesium chloride solution. Examples of the aqueous metal hydroxide solution include an aqueous sodium hydroxide solution, an aqueous potassium hydroxide solution, an aqueous calcium hydroxide solution, and an aqueous magnesium hydroxide solution. For example, when an aqueous sodium chloride solution is electrolyzed, chlorine gas is generated on the anode 24 side, hydrogen gas is generated on the cathode 25 side, and an aqueous sodium hydroxide solution is generated. A portion of the chlorine gas generated on the anode 24 side reacts with water in the aqueous solution to form hydrochloric acid and hypochlorous acid (Cl₂+H₂O→HCl+HClO).

Next, in the reaction vessel 3, the liquid transfer part 16 transfers the aqueous metal hydroxide salt solution to the reaction vessel 3, the gas transfer part 19 supplies a carbon dioxide-containing gas into the aqueous metal hydroxide salt solution in the reaction vessel 3, and carbon dioxide in the carbon dioxide-containing gas reacts with a metal salt in the aqueous metal hydroxide salt solution to generate a carbonate (reaction process). Examples of the carbonate include sodium carbonate, potassium carbonate, calcium carbonate, and magnesium carbonate. The carbonate can include bicarbonate. Therefore, examples of the carbonate include sodium bicarbonate, potassium bicarbonate, calcium bicarbonate, and magnesium bicarbonate. By this process, carbon dioxide contained in the carbon dioxide-containing gas is taken into the carbonate and separated from the carbon dioxide-containing gas.

Note that the method of bringing the aqueous metal hydroxide salt solution into contact with the carbon dioxide-containing gas is, for example, a bubbling method in which the carbon dioxide-containing gas is released into the aqueous metal hydroxide salt solution as bubbles. According to such a method, the reaction between carbon dioxide and metal salt can be made efficient. Also, the contact method may be a method in which the aqueous metal hydroxide salt solution is atomized and the carbon dioxide-containing gas is supplied into it. According to this method, the chemical reaction can be made efficient, as in the case of bubbling. In addition, the atomization method can reduce the amount of aqueous metal hydroxide salt solution to be used, and also has the effect of saving water.

As described above, the chlorine gas generated by electrolysis in the electrolysis vessel 2 reacts with water in the aqueous solution to form hydrochloric acid and hypochlorous acid. However, not all of the chlorine gas reacts with water, and some remains as chlorine gas. In FIG. 1, chlorine gas is accumulated in the upper part of the anode electrolysis chamber 20. Therefore, the gas transfer part 22 supplies the chlorine gas generated in the anode electrolysis chamber 20 to the aqueous metal salt solution in the anode electrolysis chamber 20 in the electrolysis vessel 2 (chlorine gas supply process). The gas transfer part 22 discharges, for example, the chlorine gas from the space where chlorine gas is accumulated in the upper part of the anode electrolysis chamber 20 in FIG. 1, and releases the chlorine gas into the aqueous metal salt solution in the lower part of the anode electrolysis chamber 20. The released chlorine gas reacts, for example, with water in the aqueous solution to form hydrochloric acid and hypochlorous acid (Cl₂+H₂O→HCl+HClO).

The apparatus 1A of the present embodiment can prevent the chlorine gas from being discharged to the outside by promoting the reaction with water as described above. Further, as a result of preventing the generated chlorine gas from being discharged to the outside, the necessity of suppressing the electrolysis is reduced, and therefore, this process has an effect of improving the efficiency of the electrolysis.

### Second Embodiment

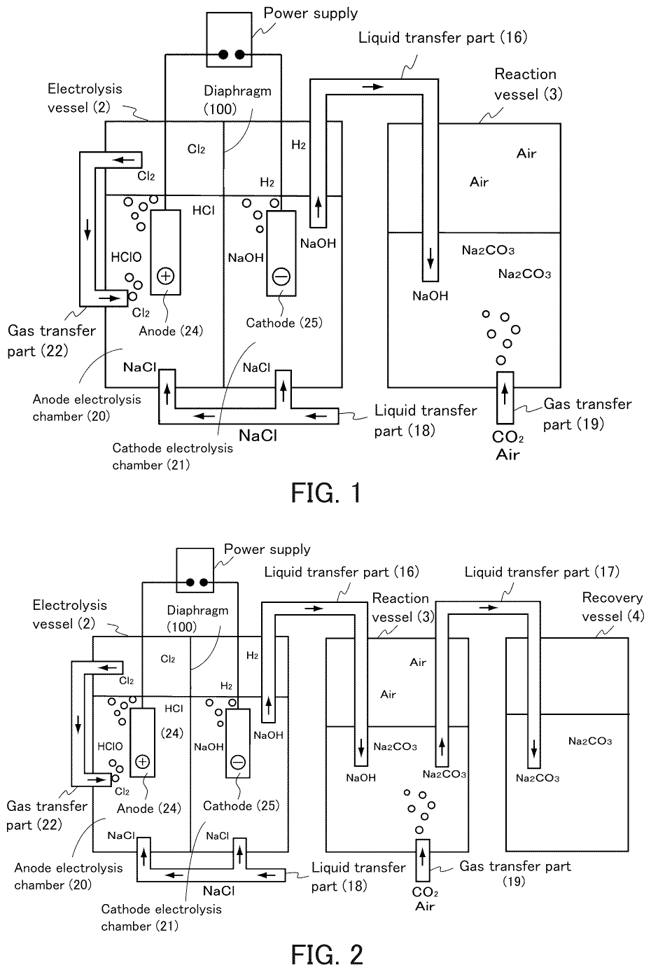

With reference to FIG. 2, an example of the configuration of the carbon dioxide recovery apparatus 1B of the present embodiment (hereinafter also referred to as the “apparatus 1B”) will be described. As shown in FIG. 2, the apparatus 1B includes, in addition to the configuration of the apparatus 1A of the first embodiment (electrolysis vessel 2 and reaction vessel 3), for example, a recovery vessel 4. The configuration of the apparatus 1A also applies to the apparatus 1B of the present embodiment, and therefore, the description provided for the first embodiment can be referred to as appropriate.

As shown in FIG. 2, the apparatus 1B of the present embodiment includes, for example, a recovery vessel 4. Further, for example, a liquid transfer part 17 is configured to supply a carbonate-containing liquid containing the carbonate generated in the reaction vessel 3 to the recovery vessel 4. The shape, material, and the like of the recovery vessel 4 are not particularly limited as similarly discussed for the electrolysis vessel 2 and the reaction vessel 3. As similarly discussed for the first embodiment, the electrolysis vessel 2, the reaction vessel 3, and the recovery vessel 4 may either be provided independently or integrally. Furthermore, the apparatus 1B may include at least one electrolysis vessel 2, at least one reaction vessel 3, and at least one recovery vessel 4, and any number of electrolysis vessels 2, reaction vessels 3, and recovery vessels 4 may be provided. The shape, material, and the like of the liquid transfer part 17 are not particularly limited, as similarity described for other liquid transfer parts.

Next, an example of the carbon dioxide recovery method of the present embodiment will be described. The carbon dioxide recovery method of the present embodiment is performed as follows, for example, using the apparatus 1B shown in FIG. 2. The carbon dioxide recovery method of the present embodiment is not limited to the use of the apparatus 1B shown in FIG. 2. Further, the order in which the following processes are performed is not limited to the order described. Therefore, the carbon dioxide recovery method of the present embodiment may be performed, for example, in an order different from the order described, or two or more processes may be performed simultaneously.

First, the electrolysis process, reaction process, and chlorine gas supply process are the same as in the first embodiment, so the descriptions of these can be referred to.

Next, the liquid transfer part 17 of the present embodiment supplies a carbonate-containing liquid containing the carbonate generated in the reaction vessel 3 to the recovery vessel 4 (recovery process). Then, as described below, when the carbonate contained in the carbonate-containing liquid supplied to the recovery vessel 4 is water-soluble, such as sodium carbonate, the apparatus 1B can extract carbon dioxide as a gas by adding, for example, hydrochloric acid in the recovery vessel 4 (Na₂CO₃+2HCl→2NaCl+H₂O+CO₂). In addition, for example, when the carbonate is sodium carbonate, the apparatus 1B can generate poorly soluble calcium carbonate by adding, for example, calcium chloride in the recovery vessel 4, thereby recovering carbon dioxide as solid calcium carbonate (Na₂CO₃+CaCl₂→2NaCl+CaCO₃). In this way, the apparatus 1B can further recover carbon dioxide from the carbonate-containing liquid in the recovery vessel 4. It should be noted that the recovery method described above is merely an example, and the present disclosure is not limited thereto. By transferring the liquid to the recovery vessel 4, the above-described recovery method can be performed, for example, in the recovery vessel 4 under the condition that the concentration of sodium hydroxide used in the reaction vessel 3 is kept low. As a result, for example, the formation of Ca(OH)₂ is suppressed when seawater containing calcium chloride is used. Therefore, the recovery method can effectively generate CaCO₃, and obtain the effect of improving carbon dioxide fixation efficiency. Therefore, in the configuration in which liquid is transferred to the recovery vessel 4, the aqueous sodium hydroxide solution or aqueous calcium hydroxide solution used in the apparatus 1B can be used in a high concentration state in the reaction vessel 3. Therefore, the concentration of each aqueous solution is not limited, and is not limited to, for example, about 0.1 mol/L, and can be used at a concentration of, for example, about 1 mol/L. In addition, since the solubility of sodium carbonate in water is 22 g/100 mL (2.1 M) at 20° C. and 45 g/100 ml (4.2 M) at 100° C., the concentration of sodium hydroxide can be further increased. Therefore, for example, the apparatus 1B has the advantage that the amount of aqueous sodium hydroxide solution used can be reduced.

The apparatus 1B of the present embodiment can use, for example, high-concentration sodium hydroxide. Therefore, the use of the apparatus 1B has an effect of significantly reducing the amount of water to be used.

### Third Embodiment

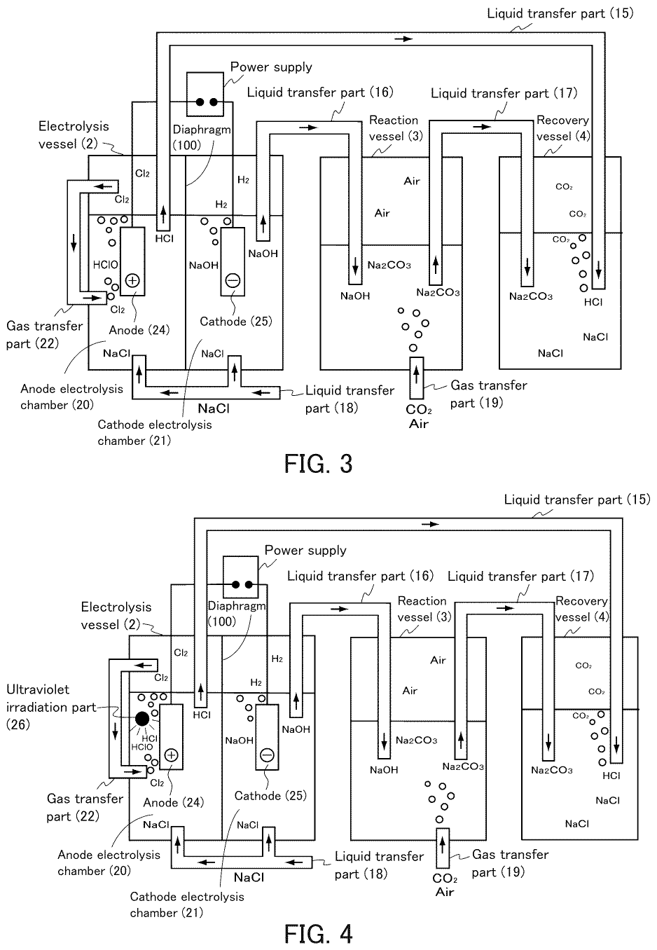

With reference to FIG. 3, an example of the configuration of the carbon dioxide recovery apparatus 1C of the present embodiment (hereinafter also referred to as the “apparatus 1C”) will be described. As shown in FIG. 3, the apparatus 1C includes, in addition to the configuration of the apparatus 1B of the second embodiment (electrolysis vessel 2, reaction vessel 3, and recovery vessel 4), for example, a liquid transfer part 15 configured to supply the aqueous hydrochloric acid solution generated in the anode electrolysis chamber 20 of the electrolysis vessel 2 to the recovery vessel. The configuration of the apparatus 1B also applies to the apparatus 1C of the present embodiment, and therefore, the description provided for the second embodiment can be referred to as appropriate.

As shown in FIG. 3, the apparatus 1C of the present embodiment includes, for example, a liquid transfer part 15 configured to supply the aqueous hydrochloric acid solution generated in the anode electrolysis chamber 20 of the electrolysis vessel 2 to the recovery vessel. The shape, material, and the like of the liquid transfer part 15 are not particularly limited as similarly described for other liquid transfer parts.

Next, an example of the carbon dioxide recovery method of the present embodiment will be described. The carbon dioxide recovery method of the present embodiment is performed as follows, for example, using the apparatus 1C shown in FIG. 3. The carbon dioxide recovery method of the present embodiment is not limited to the use of the apparatus 1C shown in FIG. 3. Further, the order in which the following processes are performed is not limited to the order described. Therefore, the carbon dioxide recovery method of the present embodiment may be performed, for example, in an order different from the order described, or two or more processes may be performed simultaneously.

First, the electrolysis process, reaction process, and chlorine gas supply process of the first embodiment are the same as those in the present embodiment. Additionally, the recovery process of the second embodiment is also the same as that in the present embodiment. Therefore, the descriptions of these can be referred to.

Next, the liquid transfer part 15 of the present embodiment supplies an aqueous hydrochloric acid solution containing hydrochloric acid generated in the anode electrolysis chamber 20 to the recovery vessel 4. Then, the apparatus 1C separates carbon dioxide gas from the carbonate-containing liquid in the recovery vessel 4 by the aqueous hydrochloric acid solution (recovery process). As described above, when the carbonate contained in the carbonate-containing liquid supplied to the recovery vessel 4 is water-soluble, such as sodium carbonate, the apparatus 1C can extract carbon dioxide as a gas by adding, for example, hydrochloric acid in the recovery vessel 4 (Na₂CO₃+2HCl→2NaCl+H₂O+CO₂).

The apparatus 1C of the present embodiment can effectively utilize the aqueous hydrochloric acid solution generated during electrolysis. As a result, the use of the apparatus 1C has an effect of enabling waste-free carbon dioxide recovery.

### Fourth Embodiment

With reference to FIG. 4, an example of the configuration of the carbon dioxide recovery apparatus 1D of the present embodiment (hereinafter also referred to as the “apparatus 1D”) will be described. As shown in FIG. 4, the apparatus 1D includes, in addition to the configuration of the apparatus 1C of the third embodiment (electrolysis vessel 2, reaction vessel 3, and recovery vessel 4), for example, an ultraviolet irradiation part 26 in the anode electrolysis chamber 20 of the electrolysis vessel 2. The configuration of the apparatus 1C also applies to the apparatus 1D of the present embodiment, and therefore, the description provided for the third embodiment can be referred to as appropriate.

As shown in FIG. 4, the anode electrolysis chamber 20 of the present embodiment includes, for example, an ultraviolet irradiation part 26. The ultraviolet irradiation part 26 irradiates, for example, the aqueous hydrochloric acid solution generated in the anode electrolysis chamber 20 with ultraviolet light, and decomposes hypochlorous acid contained in the aqueous hydrochloric acid solution to generate hydrochloric acid. The structure, shape, and position of the ultraviolet irradiation part 26 are not particularly limited as long as it can effectively irradiate the aqueous hydrochloric acid solution with ultraviolet light.

Next, an example of the carbon dioxide recovery method of the present embodiment will be described. The carbon dioxide recovery method of the present embodiment is performed as follows, for example, using the apparatus 1D shown in FIG. 4. The carbon dioxide recovery method of the present embodiment is not limited to the use of the apparatus 1D shown in FIG. 4. Further, the order in which the following processes are performed is not limited to the order described. Therefore, the carbon dioxide recovery method of the present embodiment may be performed, for example, in an order different from the order described, or two or more processes may be performed simultaneously.

First, the electrolysis process, reaction process, and chlorine gas supply process of the first embodiment are the same as those in the present embodiment. Additionally, the recovery process of the second embodiment is also the same as that in the present embodiment. Furthermore, the recovery process of the third embodiment is the same as that in the present embodiment. Therefore, the descriptions of these can be referred to.

Next, the ultraviolet irradiation part 26 of the present embodiment decomposes hypochlorous acid generated in the anode electrolysis chamber 20 to generate hydrochloric acid (electrolysis process). For example, when an aqueous sodium chloride solution is electrolyzed in the electrolysis vessel 2, the apparatus 1D generates chlorine gas on the anode 24 side and hydrogen gas on the cathode 25 side to generate sodium hydroxide. The chlorine gas generated on the anode 24 side reacts with water in the aqueous solution to form hydrochloric acid and hypochlorous acid (Cl₂+H₂O→HCl+HClO). The apparatus 1D, similar to the apparatus 1C of the third embodiment, is designed to extract carbon dioxide as a gas by adding hydrochloric acid to a carbonate-containing liquid in the recovery vessel 4. As described above, since the product of chlorine gas and water contains hypochlorous acid, the concentration of hydrochloric acid is not sufficient. Therefore, the apparatus 1D of the present embodiment irradiates the aqueous hydrochloric acid solution, which is the product, with ultraviolet light to decompose the hypochlorous acid contained in the aqueous hydrochloric acid solution, thereby producing hydrochloric acid (2HClO→2HCl+O₂). The effect of the ultraviolet light irradiation has been verified by experiments. The hypochlorous acid water with an initial concentration of 1000 ppm (50 mL) had a pH of 6.66, which decreased to pH 6.30 after 15 minutes of irradiation using the ultraviolet lamp (manufactured by Chiyo), further decreased to pH 6.11 after another 15 minutes of irradiation, and then remained constant. Therefore, by irradiating with ultraviolet light, the apparatus 1D can convert hypochlorous acid into hydrochloric acid to create an aqueous solution with high hydrochloric acid concentration, and can transfer it from the anode electrolysis chamber 20 to the recovery vessel 4.

The apparatus ID of the present embodiment can increase the hydrochloric acid concentration of the aqueous hydrochloric acid solution generated during electrolysis. Therefore, the use of the apparatus 1D has the effect of efficient carbon dioxide recovery.

### Fifth Embodiment

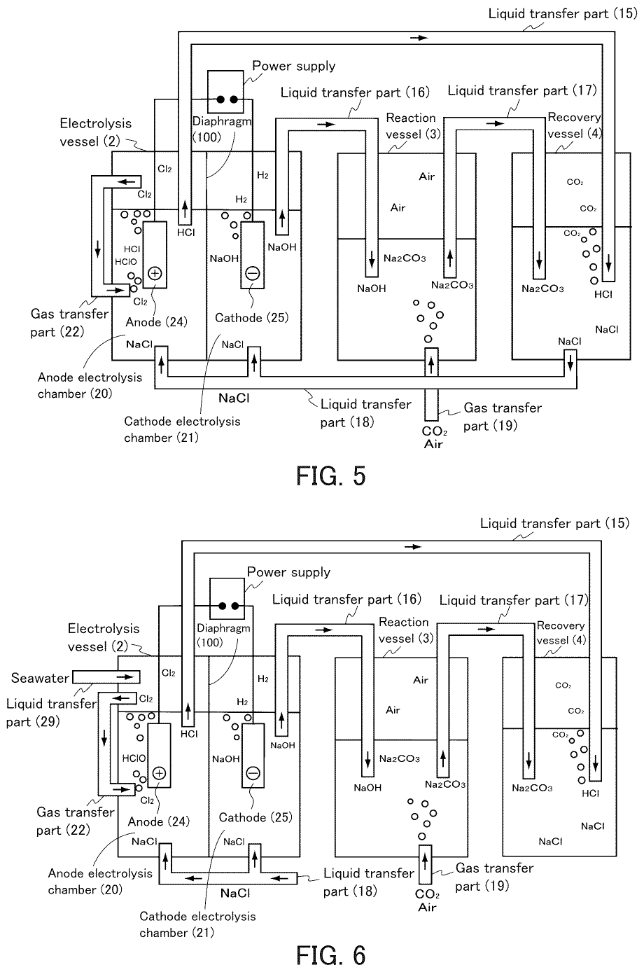

With reference to FIG. 5, an example of the configuration of the carbon dioxide recovery apparatus 1E of the present embodiment (hereinafter also referred to as the “apparatus 1E”) will be described. As shown in FIG. 5, the apparatus 1E includes, in addition to the configuration of the apparatus 1C of the third embodiment (electrolysis vessel 2, reaction vessel 3, and recovery vessel 4), for example, a liquid transfer part 18 configured to transfer the aqueous metal salt solution generated in the recovery vessel 4 to the electrolysis vessel 2. The configuration of the apparatus 1C also applies to the apparatus 1E of the present embodiment, and therefore, the description provided for the third embodiment can be referred to as appropriate. In addition, the apparatus 1E may include the configuration of the apparatus 1D of

As shown in FIG. 5, the apparatus 1E of the present embodiment includes, for example, a liquid transfer part 18 configured to transfer the aqueous metal salt solution generated in the recovery vessel 4 to the electrolysis vessel 2. The shape, material, and the like of the liquid transfer part 18 are not particularly limited as similarly described for other liquid transfer parts. Further, in FIG. 5, the liquid transfer part 18 is integrated with the liquid transfer part 18 configured to supply the aqueous metal salt solution to the electrolysis vessel 2 of the first embodiment, but the present disclosure is not limited thereto. Therefore, a liquid transfer part configured to supply the aqueous metal salt solution from the outside may be provided separately from the liquid transfer part 18 in the present embodiment.

Next, an example of the carbon dioxide recovery method of the present embodiment will be described. The carbon dioxide recovery method of the present embodiment is performed as follows, for example, using the apparatus 1E shown in FIG. 5. The carbon dioxide recovery method of the present embodiment is not limited to the use of the apparatus 1E shown in FIG. 5. Further, the order in which the following processes are performed is not limited to the order described. Therefore, the carbon dioxide recovery method of the present embodiment may be performed, for example, in an order different from the order described, or two or more processes may be performed simultaneously.

First, the electrolysis process, reaction process, and chlorine gas supply process of the first embodiment are the same as those in the present embodiment. Additionally, the recovery process of the second embodiment is also the same as that in the present embodiment. Furthermore, the recovery process of the third embodiment is also the same as that in the present embodiment. Therefore, the descriptions of these can be referred to. In addition, when the configuration of the fourth embodiment is included, the description provided for the fourth embodiment can also be referred to.

Next, the liquid transfer part 18 of the present embodiment supplies an aqueous metal salt solution generated by decomposing carbon dioxide gas from the carbonate-containing liquid in the recovery vessel 4 to at least one of the anode electrolysis chamber 20 and the cathode electrolysis chamber 21 (aqueous metal salt solution supply process). The recovery vessel 4 generates carbon dioxide gas and generates an aqueous metal salt solution through the reaction between the carbonate-containing liquid and hydrochloric acid. For example, when the carbonate-containing liquid is sodium carbonate, the recovery vessel 4 generates carbon dioxide and generates sodium chloride (Na₂CO₃+2HCl→2NaCl+H₂O+CO₂). The aqueous metal salt solution thus generated is transferred to the electrolysis vessel 2 by the liquid transfer part 18. In FIG. 5, as in FIG. 1 that relates to the first embodiment, the electrolysis vessel 2 is completely partitioned from top to bottom by the diaphragm 100. However, as described for the first embodiment, the electrolysis vessel 2 is not limited to such configuration. For example, when the electrolysis vessel 2 is not partitioned down to the bottom, which allows the anode electrolysis chamber 20 and the cathode electrolysis chamber 21 to be in fluid communication on the lower part of the electrolysis vessel 2, the supply of the aqueous metal salt solution by the liquid transfer part 18 may be performed, for example, by a method in which the aqueous metal salt solution is injected into the lower part of the electrolysis vessel 2 and is supplied to at least one of the anode electrolysis chamber 20 and the cathode electrolysis chamber 21.

In the apparatus 1E of the present embodiment, the aqueous solution after carbon dioxide recovery can be reused. As a result, the use of the apparatus 1E has an effect of enabling continuous or continual carbon dioxide recovery.

### Sixth Embodiment

With reference to FIG. 6, an example of the configuration of the carbon dioxide recovery apparatus 1F of the present embodiment (hereinafter also referred to as the “apparatus 1F”) will be described. The apparatus 1F includes, in addition to the configuration of each of the carbon dioxide recovery apparatuses of the first to fifth embodiments, for example, a liquid transfer part 29 configured to supply seawater to the electrolysis vessel 2. The configuration of each of the carbon dioxide recovery apparatuses of the first to fifth embodiments also applies to the apparatus 1F of the present embodiment, and therefore, the descriptions provided for the first to fifth embodiments can be referred to as appropriate. Note that FIG. 6 is a schematic diagram showing the configuration of the carbon dioxide recovery apparatus of FIG. 3 including the configuration added in the present embodiment.

As shown in FIG. 6, the apparatus 1F of the present embodiment includes, for example, a liquid transfer part 29 configured to supply seawater to the electrolysis vessel 2. The shape, material, and the like of the liquid transfer part 29 are not particularly limited, as similarly described for other liquid transfer parts.

The carbon dioxide recovery method of the present embodiment is the same as the carbon dioxide recovery methods of the first to fifth embodiments, except that it is performed, for example, by using the apparatus 1F shown in FIG. 6 and supplying seawater to the recovery vessel 2. The seawater is supplied to at least one of the anode electrolysis chamber 20 and the cathode electrolysis chamber 21 using the liquid transfer part 29. When supplying an aqueous metal salt solution in the fifth embodiment, depending on the structure of the electrolysis vessel 2, the seawater may be supplied by, for example, a method of injecting it into the lower part of the electrolysis vessel. Further, as similarly described for the other embodiments, the order in which the processes are performed is not limited.

The apparatus 1F of the present embodiment can recover carbon dioxide using seawater. When the apparatus 1F is mounted on a large tanker, the apparatus 1F can obtain electricity and hydrogen while electrolyzing seawater, fixate carbon dioxide, and generate calcium carbonate by utilizing calcium ions in seawater.

### Seventh Embodiment

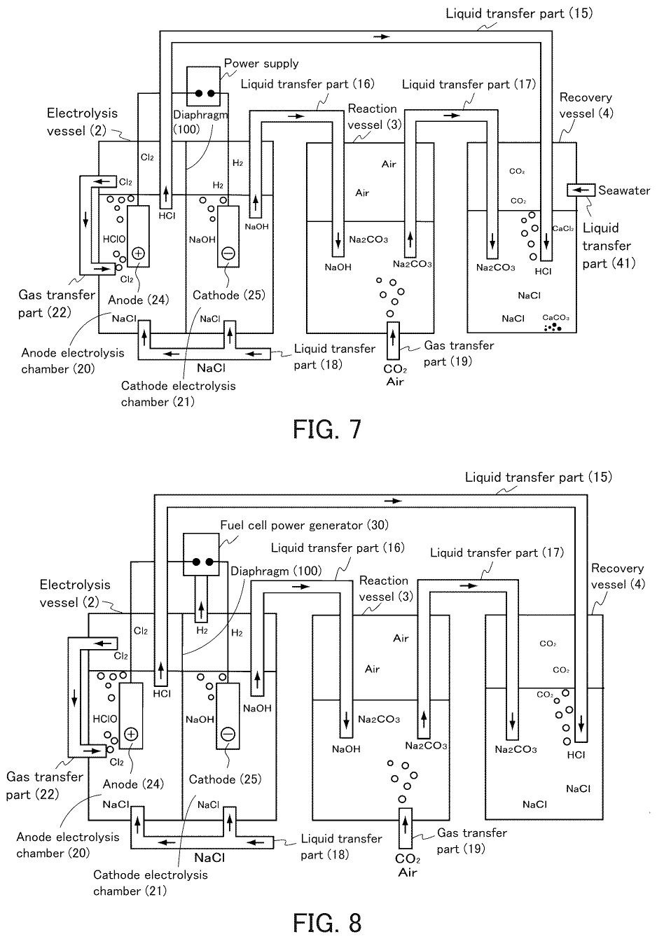

With reference to FIG. 7, an example of the configuration of the carbon dioxide recovery apparatus 1G of the present embodiment (hereinafter also referred to as the “apparatus 1G”) will be described. The apparatus 1G includes, in addition to the configuration of each of the carbon dioxide recovery apparatuses of the second and third embodiments, for example, a liquid transfer part 41 configured to supply seawater to the recovery vessel 4. The configuration of each of the carbon dioxide recovery apparatuses of the second and third embodiments also applies to the apparatus 1G of the present embodiment, and therefore, the descriptions provided for the second and third embodiments can be referred to as appropriate. Note that FIG. 7 is a schematic diagram showing the configuration of the carbon dioxide recovery apparatus of FIG. 3 including the configuration added in the present embodiment. Furthermore, the configuration of the sixth embodiment may be added to the present embodiment.

As shown in FIG. 7, the apparatus 1G of the present embodiment includes, for example, a liquid transfer part 41 configured to supply seawater to the recovery vessel 4. The shape, material, and the like of the liquid transfer part 41 are not particularly limited as similarly described for the other liquid transfer parts.

The carbon dioxide recovery method of the present embodiment is the same as the carbon dioxide recovery methods of the second and third embodiments, except that it is performed, for example, by using the apparatus 1G shown in FIG. 7 and supplying seawater to the recovery vessel 4. Further, as similarly described for the other embodiments, the order in which the processes are performed is not limited. Furthermore, each process of the sixth embodiment may be included.

In the apparatus 1G of the present embodiment, by taking seawater into the recovery vessel 4, for example, calcium carbonate is generated by a reaction between the carbonate generated in the reaction vessel 3 and calcium chloride contained in the seawater. For example, since calcium carbonate has poor solubility, it precipitates in solid form on the bottom of the recovery vessel 4. Therefore, the apparatus 1G can recover carbon dioxide by recovering the precipitate. For example, when the carbonate is sodium carbonate, the apparatus 1G can generate calcium carbonate in the recovery vessel 4 by adding calcium chloride contained in seawater, and recover carbon dioxide as solid calcium carbonate (Na₂CO₃+CaCl₂→2NaCl+CaCO₃). Note that the above examples are merely examples, and the present disclosure is not limited thereto.

As similarly described for the apparatus 1F of the sixth embodiment, when the apparatus 1G of the present embodiment is mounted on a large tanker, the apparatus 1G can obtain electricity and hydrogen while electrolyzing seawater, fixate carbon dioxide, and generate calcium carbonate by utilizing calcium ions in seawater.

### Eighth Embodiment

With reference to FIG. 8, an example of the configuration of the carbon dioxide recovery apparatus 1H of the present embodiment (hereinafter also referred to as the “apparatus 1H”) will be described. The apparatus 1H includes, in addition to the configuration of each of the carbon dioxide recovery apparatuses of the first to seventh embodiments, for example, a fuel cell power generator 30. The configuration of each of the carbon dioxide recovery apparatuses of the first to seventh embodiments also applies to the apparatus 1H of the present embodiment, and therefore, the descriptions provided for the first to seventh embodiments can be referred to as appropriate. Note that FIG. 8 is a schematic diagram showing the configuration of the carbon dioxide recovery apparatus of FIG. 3 including the configuration added in the present embodiment.

As shown in FIG. 8, the apparatus 1H of the present embodiment includes, for example, the fuel cell power generator 30 and a gas transfer part 13 configured to supply hydrogen gas generated in the cathode electrolytic chamber 21 to the fuel cell power generator 30. The fuel cell power generator 30 is configured to generate electric power by utilizing hydrogen generated during electrolysis in the cathode electrolytic chamber 21 of the electrolysis vessel 2. The fuel cell power generator 30 may also be configured to supply electricity to the anode 24 and the cathode 25 provided in the electrolysis vessel 2. The fuel cell power generator 30 may have any form and structure as long as it can generate electric power utilizing hydrogen. The shape, material, and the like of the gas transfer part 13 are not particularly limited, as similarly described for other gas transfer parts.

The carbon dioxide recovery method of the present embodiment is the same as the carbon dioxide recovery methods of the first to seventh embodiments, except that, for example, the apparatus 1H shown in FIG. 8 is used to generate electric power in the cathode electrolytic chamber 21 of the electrolysis vessel 2 by utilizing hydrogen generated during electrolysis. Further, as similarly described for other embodiments, the order in which the processes are performed is not limited.

The gas transfer part 13 of the present embodiment supplies the hydrogen gas generated in the cathode electrolytic chamber 21 to the fuel cell power generator 30 (fuel cell power generation process). The fuel cell power generator 30 generates electric power utilizing the hydrogen supplied by the gas transfer part 13. The fuel cell power generator 30 may supply the generated electricity to the anode 24 and the cathode 25 provided in the electrolysis vessel 2.

The apparatus 1H of the present embodiment can effectively utilize the hydrogen gas generated during electrolysis.

### Ninth Embodiment

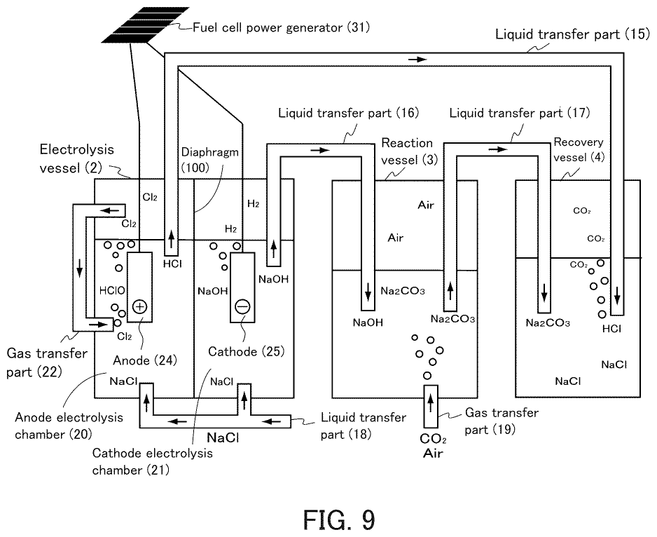

With reference to FIG. 9, an example of the configuration of the carbon dioxide recovery apparatus 1I of the present embodiment (hereinafter also referred to as the “apparatus 1I”) will be described. The apparatus 1I includes, in addition to the configuration of each of the carbon dioxide recovery apparatuses of the first to eighth embodiments, for example, a solar power generator 31. The configuration of each of the carbon dioxide recovery apparatuses of the first to eighth embodiments also applies to the apparatus 1I of the present embodiment, and therefore, the descriptions provided for the first to eighth embodiments can be referred to as appropriate. Note that FIG. 9 is a schematic diagram showing the configuration of the carbon dioxide recovery apparatus of FIG. 3 including the configuration added in the present embodiment.

As shown in FIG. 9, the apparatus 1I of the present embodiment includes, for example, the solar power generator 31 configured to supply electricity to the anode 24 and the cathode 25. The solar power generator 31 may have any form and structure as long as it can supply electricity to the anode 24 and the cathode 25.

In the carbon dioxide recovery method of the present embodiment, for example, electricity is supplied to the anode 24 and the cathode 25 provided in the electrolysis vessel 2 by using the apparatus 1I shown in FIG. 9. The carbon dioxide recovery method of the present embodiment is the same as the carbon dioxide recovery methods of the first to eighth embodiments except for the above-described points. Further, as similarly described for the other embodiments, the order in which the processes are performed is not limited.

The apparatus 1I of the present embodiment enables carbon dioxide recovery utilizing renewable energies.

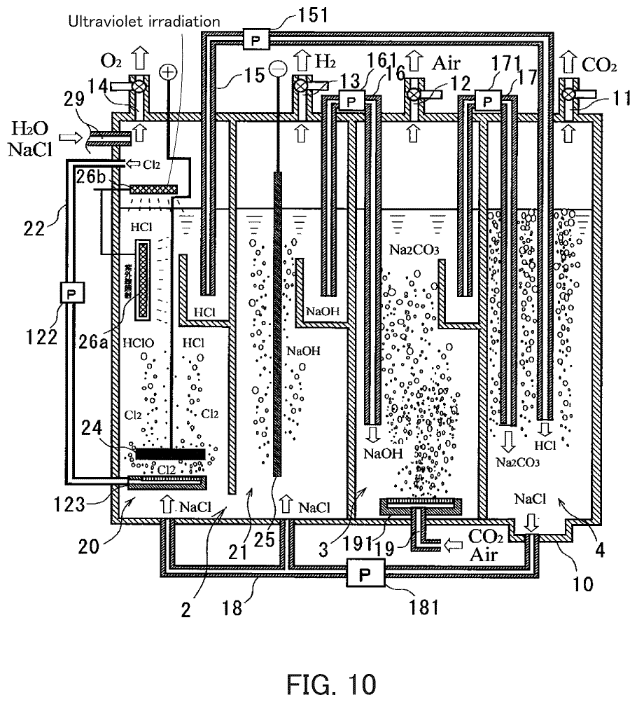

Hereinafter, a specific example of the carbon dioxide recovery apparatus and the carbon dioxide recovery method according to the present disclosure will be described with reference to FIG. 10. Note that the following description is an example of specific details, and as described above, the carbon dioxide recovery apparatus and the carbon dioxide recovery method according to the present disclosure are not limited to the following description. In addition, the carbon dioxide recovery apparatus and the carbon dioxide recovery method according to the present disclosure do not necessarily include all of the configurations described below, and may also include additional configuration. Further, the carbon dioxide recovery method according to the present disclosure is not limited to the use of the carbon dioxide recovery apparatus (hereinafter, also referred to as the “apparatus 1J”) described below.

The apparatus 1J is an apparatus which introduces air containing carbon dioxide from an air introduction path (gas transfer part 19), separates carbon dioxide to make the concentration of carbon dioxide zero or greatly reduced, and discharges it from an air discharge path (gas transfer part 12) to the outside. It is assumed to be applied to facilities of various scales, from large-scale facilities such as thermal power plants which continuously discharge a large amount of combustion gas to small-scale facilities such as houses and offices.

The apparatus 1J includes, inside a hollow rectangular parallelepiped housing 10, an electrolysis vessel 2 including an anode 24 and a cathode 25 configured to generate aqueous sodium hydroxide solution and an aqueous solution containing hydrochloric acid while electrolyzing introduced aqueous sodium chloride solution; a reaction vessel 3 configured to introduce the aqueous sodium hydroxide solution generated on the cathode 25 side through a pipe (liquid transfer part 16) and introduce air containing carbon dioxide through an air introduction path (gas transfer part 19) to jet and bubble it from a bubble generator 191 in the aqueous sodium hydroxide solution, thereby producing an aqueous solution containing sodium carbonate and sodium bicarbonate; and a recovery vessel 4 configured to introduce the generated aqueous solution containing sodium carbonate and the like through a pipe (liquid transfer part 17) and introduce an aqueous solution containing hydrochloric acid through a pipe (liquid transfer part 15) to mix with it, thereby extracting gaseous carbon dioxide from a carbon dioxide discharge path (gas transfer part 11).

The apparatus 1J includes ultraviolet lamps (ultraviolet irradiation parts 26a, 26b) as ultraviolet irradiation parts installed in the upper side of the area where the aqueous solution containing hydrochloric acid is stored in the electrolysis vessel 2, and is configured to decompose hypochlorous acid contained in the aqueous solution electrolyzed on the anode 24 side to generate hydrochloric acid, thereby increasing the concentration of hydrochloric acid before introducing it into the recovery vessel 4 through a pipe (liquid transfer part 15).

That is, for example, by adopting a configuration where the aqueous solution containing hydrochloric acid generated on the anode 24 side of the electrolysis vessel 2 is irradiated with ultraviolet light using ultraviolet lamps (ultraviolet irradiation parts 26a, 26b), it becomes possible to convert the hypochlorous acid contained in the aqueous solution into hydrochloric acid, thereby obtaining an aqueous solution with high hydrochloric acid concentration and low pH, and introducing the solution into the recovery vessel 4. Therefore, the apparatus 1J efficiently separates carbon dioxide in gas form while producing an aqueous sodium chloride solution for recirculation. Furthermore, by separately providing the reaction vessel 3 and the recovery vessel 4, the apparatus 1J enables continuous processing in the vessels while allowing the aqueous solutions used for processing to be recirculated without replacement operations, thereby achieving high operational efficiency.

On the other hand, in the electrolysis vessel 2, the anode 24 side and cathode 25 side are partitioned by a vertically extending diaphragm 100 from near the bottom in the vertical direction up to the top wall, forming the anode electrolysis chamber 20 and the cathode electrolysis chamber 21, with spaces on the upper side for storing gases, and an aqueous sodium chloride solution (brine) generated in the recovery vessel 4 is introduced from the bottom side of the recovery vessel 4 to the bottom sides of both the anode electrolysis chamber 20 and the cathode electrolysis chamber 21 through a pipe (liquid transfer part 18).

A suction port of a pipe (liquid transfer part 16) configured to transfer the aqueous sodium hydroxide solution in the cathode electrolysis chamber 21 to the reaction vessel 3 and a suction port of a pipe (liquid transfer part 15) configured to transfer the aqueous solution containing hydrogen chloride in the anode electrolysis chamber 20 to the recovery vessel 4 are each opened on the upper side of the aqueous solution storage area. As a result, this apparatus can continuously introduce the aqueous sodium chloride solution generated in the recovery vessel 4 into the electrolysis vessel 2 while minimizing the introduction of non-electrolyzed aqueous sodium chloride solution mixed with electrolyzed aqueous solution into the next vessel, making continuous operation of the apparatus possible.

In the above-described electrolysis vessel 2, heat is generated on the surfaces of the anode 24 and cathode 25 during electrolysis, causing the temperature to rise in both the aqueous solution containing hypochlorous acid and hydrogen chloride and the aqueous solution containing sodium hydroxide generated by electrolysis. Additionally, with the irradiation heat from the ultraviolet lamps (ultraviolet irradiation parts 26a, 26b) disposed at the upper part on the anode 24 side, thermal expansion causes these aqueous solutions to rise more readily to the upper part of the vessel. Therefore, the apparatus 1J further enhances the state where a non-electrolyzed aqueous sodium chloride solution is less likely to be mixed with the electrolyzed aqueous solution and sucked in from the suction ports of the pipes (liquid transfer parts 15, 16). In addition, the diaphragm 100 which partitions the space of the electrolysis vessel 2 into right and left sides is made of a hard impermeable material and its lower end may be opened to allow the anode electrolysis chamber 20 and the cathode electrolysis chamber 21 to be in fluid communication, but a semipermeable membrane may also be used for the diaphragm 100, in which case the right and left spaces of the electrolysis vessel 2 may be completely separated.

Also, a pipe (gas transfer part 22) is connected to the anode electrolysis chamber 20 of the apparatus 1J, extending from the upper space where gas is stored to the lower part of the area where an aqueous solution is stored. Chlorine gas generated by electrolysis and accumulated in the upper space is transferred by a pump 122 to the lower part and bubbled in the aqueous solution using a bubble generator 123, thereby dissolving the chlorine gas in the aqueous solution.

Therefore, the apparatus 1J can repeatedly dissolve dangerous chlorine gas, generated by electrolysis on the anode 24 side and accommodated in the upper space, in the stored aqueous solution by ejecting the chlorine gas from the lower side of the stored aqueous solution, and can convert most of the chlorine gas to a relatively safe liquid state (hydrochloric acid). This enables the apparatus 1J to perform electrolysis with sufficient output without giving special consideration to the amount of chlorine gas generated while ensuring safety of the apparatus, and generate sodium hydroxide with sufficient concentration on the cathode 25 side.

The top wall of the anode electrolysis chamber 20 is provided with an oxygen discharge path (gas transfer part 14) configured to discharge oxygen, and the top wall of the cathode electrolysis chamber 21 is provided with a hydrogen discharge path (gas transfer part 13) configured to discharge hydrogen. The discharged oxygen and hydrogen can be stored in predetermined cylinders and used for various purposes. For example, although it is not shown, the present apparatus may be provided with a fuel cell power generator using hydrogen gas as a fuel for power generation, so that electric power generated by the fuel cell power generator can be used as at least a part of electric power for operating the apparatus. In this case, it is possible to further reduce the running cost of the apparatus. The present apparatus may also utilize solar power generation. This makes it possible to further reduce running costs.

Although it is not shown, in the apparatus 1J, by providing each vessel with a pH meter and an electronic controller having a microcomputer and a memory storing operation control software, and having that the electronic controller operates the pumps 151, 161, 171, 181, and 122 provided in the pipes (liquid transfer parts 15, 16, 17, and 18, and gas transfer part 22), the electromagnetic valves provided in the pathes, and the ultraviolet lamps (ultraviolet irradiation parts 26a, 26b) by feedback control while detecting the pH level of each vessel, the apparatus 1J can be operated almost automatically and continuously.

When operating the apparatus 1J for the first time, it is advisable to store an aqueous sodium chloride solution (brine) having a predetermined concentration up to a predetermined liquid level in the electrolysis vessel 2 through a brine introduction path (liquid transfer part 29), as well as to introduce an aqueous sodium hydroxide solution in the reaction vessel 3 and store it up to a predetermined level using the air discharge path (gas transfer part 12) and the carbon dioxide discharge path (gas transfer part 11), and to introduce an aqueous sodium chloride solution into the recovery vessel 4 and store it up to a predetermined liquid level.

Next, the apparatus 1J starts electrolysis by applying current to the anode 24 and cathode 25, while turning on the ultraviolet lamps (ultraviolet irradiation parts 26a, 26b) of the anode electrolysis chamber 20 and driving the pump 122 of the pipe (gas transfer part 22). At the same time, the apparatus 1J begins introducing air containing carbon dioxide (the treatment target) into the reaction vessel 3 through the air introduction path (gas transfer part 19) and the bubble generator 191.

Then, when a pH meter (not shown) disposed at the upper side of the solution storage area of the cathode electrolysis chamber 25 detects that the basicity of the solution has reached a predetermined level, the apparatus 1J drives the pump 161 of the pipe (liquid transfer part 16) to introduce an aqueous sodium hydroxide solution into the reaction vessel 3, and when a pH meter (not shown) disposed at the upper side of the solution storage area of the anode electrolysis chamber 24 detects that the acidity of the solution has reached a predetermined level, the apparatus 1J drives the pump 151 of the pipe (liquid transfer part 15) to introduce an aqueous hydrochloric acid solution (hydrochloric acid) into the recovery vessel 4.

On the other hand, when a pH meter (not shown) disposed at the upper side of the aqueous solution storage area of the reaction vessel 3 detects that the basicity of the aqueous solution has decreased to a predetermined level, the apparatus 1J drives the pump 171 of the pipe (liquid transfer part 17) to introduce an aqueous solution containing sodium carbonate and sodium bicarbonate into the recovery vessel 4, and reacts it with hydrogen chloride introduced through the pipe (liquid transfer part 15) to generate sodium chloride, while carbon dioxide generated by the reaction separates in gas form and accumulates in the upper space. Then, the separated carbon dioxide is discharged to the outside through the carbon dioxide discharge path (gas transfer part 11) and stored in a predetermined storage device, while the remaining aqueous sodium chloride solution is sent out by driving the pump 181 of the pipe (liquid transfer part 18) by the amount corresponding to the rise in the liquid level, introduced from the bottom of the electrolysis vessel 2, and reused.

Thereafter, in the apparatus 1J, the aqueous solution in each vessel is circulated, generated, and used, making it possible to continue operating the apparatus with almost no replenishment of materials, thereby keeping the running costs of the apparatus low. On the other hand, since the liquid level decreases due to hydrogen and oxygen discharged by electrolysis and water evaporation, water replenishment to the apparatus is performed as appropriate.

The reaction in which hypochlorous acid generated together with hydrogen chloride on the anode 24 side by electrolysis is decomposed into hydrogen chloride by ultraviolet irradiation (2HClO→2HCl+O₂) has been verified by experiments conducted by the present inventors. That is, the hypochlorous acid water with an initial concentration of 1000 ppm (50 mL) had a pH of 6.66, which decreased to pH 6.30 after 15 minutes of irradiation using the ultraviolet lamp (manufactured by Chiyo), further decreased to pH 6.11 after another 15 minutes of irradiation, and then remained constant. Therefore, it can be seen that in the anode electrolysis chamber 20 of the apparatus 1J, the hypochlorous acid in the solution storage area is converted into hydrochloric acid by irradiation with the ultraviolet lamps (ultraviolet irradiation parts 26a and 26b), and the aqueous solution with a high hydrochloric acid concentration and low pH is discharged.

It should be noted that, while the apparatus 1J has been described with reference to a case where sodium carbonate and sodium bicarbonate are generated by bubbling carbon dioxide-containing air in the introduced aqueous sodium hydroxide solution in the reaction vessel 3, similar results can be achieved by atomizing the introduced aqueous sodium hydroxide solution and mixing it with carbon dioxide-containing air. In addition, instead of using an aqueous sodium chloride solution, an aqueous potassium chloride solution can be used to perform the same process.

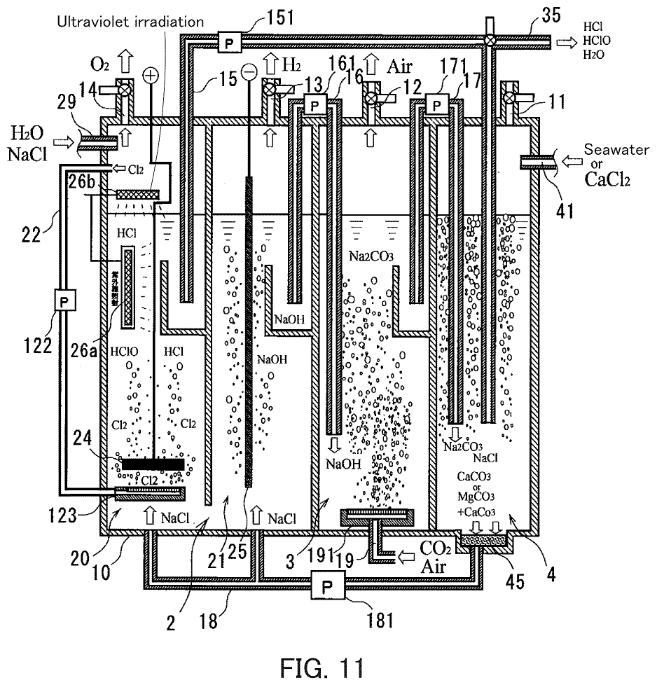

Hereinafter, another specific example of the carbon dioxide recovery apparatus according to the present disclosure will be described with reference to FIG. 11. Note that the following description is an example of specific details, and as described above, the carbon dioxide recovery apparatus and the carbon dioxide recovery method according to the present disclosure are not limited to the following description.

The carbon dioxide recovery apparatus described below (hereinafter also referred to as the “apparatus 1K”) utilizes the same configuration as the previously described specific example, but additionally includes a hydrochloric acid discharge path (liquid transfer part 35) that branches through an electromagnetic valve from the pipe (liquid transfer part 15) and configured to discharge an aqueous solution containing hydrochloric acid and hypochlorous acid, a seawater introduction path (liquid transfer part 41) installed on the upper side of the recovery vessel 4 and configured to introduce seawater, and a filter 45 added to the bottom side to capture solid materials that contain fixated carbon dioxide.

The apparatus 1K of FIG. 11 is intended to recover carbon dioxide in solid form, rather than separating it in gas form for storage or use outside the apparatus. That is, the aqueous solution containing hydrogen chloride generated in the anode electrolysis chamber 20 is not used and is discharged from the hydrochloric acid discharge path (liquid transfer part 35), and instead, seawater or calcium chloride (CaCl₂) is introduced into the recovery vessel 4 from the seawater introduction path (liquid transfer part 41). That is, in the recovery vessel 4, what is added to sodium carbonate (Na₂CO₃) and sodium bicarbonate (NaHCO₃) (carbonate) is hydrochloric acid (HCl) in the apparatus 1J in the above-mentioned embodiment, but is seawater or calcium chloride (CaCl₂) in the apparatus 1K in the present embodiment. When seawater is added, for example, magnesium carbonate (MgCO₃) and calcium carbonate (CaCO₃) are obtained, and when calcium chloride (CaCl₂) is added, calcium carbonate (CaCO₃) is obtained. The compounds (magnesium carbonate, calcium carbonate, and the like) generated in the recovery vessel 4 precipitate in the aqueous solution in solid form. As a result, the apparatus 1K can recover carbon dioxide as a solid compound. In addition, since calcium carbonate is extremely insoluble in water and harmless, it enables safe and reliable recovery of carbon dioxide.

In the conventional technology in which carbon dioxide is fixated by electrolysis of seawater, since a large amount of magnesium hydroxide (Mg(OH)₂) is first generated by direct treatment with an aqueous sodium hydroxide solution, the treatment of carbonate-containing liquid with seawater does not progress, and the recovery of carbon dioxide as a compound precipitated in the aqueous solution is hindered. In the apparatus 1K, since the treatment is performed with the carbonate-containing liquid generated as a result of mixing carbon dioxide and sodium hydroxide, the above-described problem does not occur.

For example, in the conventional carbon dioxide fixation utilizing an aqueous sodium hydroxide solution and calcium ions, when the concentration of sodium hydroxide is high, as described above, the aqueous sodium hydroxide solution and calcium ions are likely to react with each other first, hindering the fixation of carbon dioxide. Therefore, the concentration of sodium hydroxide needs to be, for example, 0.2 mol/L or less. Therefore, the conventional fixation of carbon dioxide requires a large amount of water. In contrast, in the carbon dioxide recovery method according to the present disclosure, as described above, the concentration of sodium hydroxide can be increased because the problem of the conventional method does not occur. For example, the concentration may be 1 mol/L, but is not limited thereto. As a result, the amount of water used for carbon dioxide fixation can be significantly reduced. Further, in the treatment in the reaction vessel 3, the amount of water can be further reduced by the above-mentioned method of atomizing sodium hydroxide. Furthermore, in order to enlarge the reaction space of the reaction vessel 3, it is possible to fixate a large amount of carbon dioxide at low cost by using an empty warehouse, a huge dome, a cave, a tunnel, or an abandoned mine.

When the carbon dioxide recovery apparatus according to the present disclosure is mounted on a large tanker, the carbon dioxide recovery apparatus can obtain electricity and hydrogen while electrolyzing seawater, fixate carbon dioxide, and generate calcium carbonate by utilizing calcium ions in seawater. By combining the use of hydrogen gas generated during electrolysis and solar power generation, more stable carbon dioxide recovery can be performed even on a large tanker. Furthermore, since the generated calcium carbonate is extremely insoluble and harmless as described above, it can be discharged into the deep sea. While transportation and storage of the recovered carbon dioxide are regarded as a problem, the carbon dioxide recovery apparatus according to the present disclosure can solve this problem. Furthermore, if vast land that has not been used due to a nuclear accident is used instead of a tanker, it is possible to contribute to the solution of the global warming problem and to revitalize the area damaged by the nuclear accident.

### INDUSTRIAL APPLICABILITY

As described above, according to the present disclosure, the carbon dioxide recovery apparatus can continuously and efficiently recover carbon dioxide from a gas containing carbon dioxide while keeping the running cost low. Therefore, the present invention is particularly useful for global warming mitigation, and the like.

This application claims priority from Japanese Patent Application No. 2022-199049 filed on Dec. 14, 2022 and Japanese Patent Application No. 2023-073210 filed on Apr. 27, 2023. The entire subject matters of the above Japanese Patent Applications are incorporated herein by reference.

### REFERENCE SIGNS LIST

1A to 1K: carbon dioxide recovery apparatus
2: electrolysis vessel
3: reaction vessel
4: recovery vessel
10: housing
11 to 14, 19, and 22: gas transfer part
15 to 18, 29, 35, and 41: liquid transfer part
20: anode electrolysis chamber
21: cathode electrolysis chamber
24: anode
25: cathode
26a, 26b: ultraviolet irradiation part
30: fuel cell power generator
31: solar power generator
100: diaphragm
122, 151, 161, 171, 181: pump

1. A carbon dioxide recovery apparatus, comprising:
an electrolysis vessel;
a reaction vessel;
a liquid transfer part; and
a gas transfer part, wherein
the electrolysis vessel comprises a diaphragm, an anode, and a cathode,
an anode electrolysis chamber and a cathode electrolysis chamber are provided in the electrolysis vessel by the diaphragm,
the anode is disposed in the anode electrolysis chamber,
the cathode is disposed in the cathode electrolysis chamber,
the liquid transfer part is configured to supply an aqueous metal salt solution to both the anode electrolysis chamber and the cathode electrolysis chamber,
an aqueous metal hydroxide salt solution is generated by electrolysis in the cathode electrolysis chamber,
the liquid transfer part is configured to transfer the aqueous metal hydroxide salt solution to the reaction vessel,
the gas transfer part is configured to supply carbon dioxide-containing gas to the aqueous metal hydroxide salt solution in the reaction vessel, such that carbon dioxide in the carbon dioxide-containing gas reacts with a metal salt in the aqueous metal hydroxide salt solution to generate a carbonate, and
the gas transfer part is configured to supply chlorine gas generated in the anode electrolysis chamber to the aqueous metal salt solution in the anode electrolysis chamber in the electrolysis vessel.

2. The carbon dioxide recovery apparatus according to claim 1, further comprising:
a recovery vessel, wherein
the liquid transfer part is configured to supply a carbonate-containing liquid containing the carbonate to the recovery vessel.

3. The carbon dioxide recovery apparatus according to claim 2, wherein
the liquid transfer part is configured to supply an aqueous hydrochloric acid solution containing hydrochloric acid generated in the anode electrolysis chamber to the recovery vessel, and
carbon dioxide gas is separated from the carbonate-containing liquid by the aqueous hydrochloric acid solution.

4. The carbon dioxide recovery apparatus according to claim 3, wherein
the anode electrolysis chamber comprises an ultraviolet irradiation part, and
the ultraviolet irradiation part is configured to decompose hypochlorous acid generated in the anode electrolysis chamber to generate the hydrochloric acid.

5. The carbon dioxide recovery apparatus according to claim 3, wherein
the liquid transfer part is configured to supply the aqueous metal salt solution that is generated by separating the carbon dioxide gas from the carbonate-containing liquid in the recovery vessel to at least one chamber selected from the group consisting of the anode electrolysis chamber and the cathode electrolysis chamber.

6. The carbon dioxide recovery apparatus according to claim 1, wherein
the liquid transfer part is configured to supply seawater to at least one chamber selected from the group consisting of the anode electrolysis chamber and the cathode electrolysis chamber.

7. The carbon dioxide recovery apparatus according to claim 2, wherein
the liquid transfer part is configured to supply seawater to the recovery vessel.

8. The carbon dioxide recovery apparatus according to claim 1, further comprising:
a fuel cell power generator, wherein
the gas transfer part is configured to supply hydrogen gas generated in the cathode electrolysis chamber to the fuel cell power generator.

9. The carbon dioxide recovery apparatus according to claim 8, wherein
the fuel cell power generator is configured to supply electricity to the anode and the cathode.

10. The carbon dioxide recovery apparatus according to claim 1, further comprising:
a solar power generator configured to supply electricity to the anode and the cathode.

11. A carbon dioxide recovery method, comprising:
electrolyzing;
reacting; and
supplying chlorine gas, wherein
the electrolyzing is performed using an electrolysis vessel,
the electrolysis vessel comprises a diaphragm, an anode, and a cathode,
an anode electrolysis chamber and a cathode electrolysis chamber are provided in the electrolysis vessel by the diaphragm,
the anode is disposed in the anode electrolysis chamber,
the cathode is disposed in the cathode electrolysis chamber,
the electrolyzing includes supplying an aqueous metal salt solution to both the anode electrolysis chamber and the cathode electrolysis chamber, and generating an aqueous metal hydroxide salt solution by electrolysis in the cathode electrolysis chamber,
the reacting is performed using a reaction vessel,
the reacting includes transferring the aqueous metal hydroxide salt solution is to the reaction vessel, supplying carbon dioxide-containing gas to the aqueous metal hydroxide salt solution in the reaction vessel, and reacting carbon dioxide in the carbon dioxide-containing gas with a metal salt in the aqueous metal hydroxide salt solution to generate carbonate, and
the supplying of the chlorine gas includes supplying the chlorine gas generated in the anode electrolysis chamber to the aqueous metal salt solution in the anode electrolysis chamber in the electrolysis vessel.

12. The carbon dioxide recovery method according to claim 11, further comprising:
recovering, wherein
the recovering is performed using a recovery vessel, and
the recovering includes supplying a carbonate-containing liquid containing the carbonate to the recovery vessel.

13. The carbon dioxide recovery method according to claim 12, wherein
the recovering includes supplying an aqueous hydrochloric acid solution containing hydrochloric acid generated in the anode electrolysis chamber to the recovery vessel, and separating carbon dioxide gas from the carbonate-containing liquid by the aqueous hydrochloric acid solution.

14. The carbon dioxide recovery method according to claim 13, wherein
the anode electrolysis chamber comprises an ultraviolet irradiation part, and
the electrolyzing includes decomposing, with the ultraviolet irradiation part, hypochlorous acid generated in the anode electrolysis chamber to generate hydrochloric acid.

15. The carbon dioxide recovery method according to claim 13, wherein
the supplying of the aqueous metal salt solution includes:
generating the aqueous metal salt solution by separating the carbon dioxide gas from the carbonate-containing liquid in the recovery vessel, and
supplying the aqueous metal salt solution to at least one chamber selected from the group consisting of the anode electrolysis chamber and the cathode electrolysis chamber.

16. The carbon dioxide recovery method according to claim 11 further comprising:
supplying seawater, to at least one chamber selected from the group consisting of the anode electrolysis chamber and the cathode electrolysis chamber.

17. The carbon dioxide recovery method according to claim 12, further comprising:
supplying seawater to a recovery vessel.

18. The carbon dioxide recovery method according to claim 11, generator, further comprising:
generating fuel cell power using a fuel cell powder generator, which includes:
supplying hydrogen gas generated in the cathode electrolysis chamber to the fuel cell power generator.

19. The carbon dioxide recovery method according to claim 18, wherein
the generating of the fuel cell power includes supplying electricity to the anode and the cathode from the fuel cell power generator.

20. The carbon dioxide recovery method according to claim 11, further comprising:
generating solar power using a solar powder generator, which includes:
supplying electricity to the anode and the cathode.
